(19) United States
(12) Patent Application Publication
(10) Pub. No.: US 2007/0214002 A1
(43) Pub. Date: Sep. 13, 2007

(54) System for outpatient treatment of chronic health conditions

(76) Inventors: James Charles Smith, Highland Park, TX (US); Melissa Antonia Cadwell, Orangevale, CA (US); Kathy Jane Corbin, Glen Ellyn, IL (US); Kelly Godfrey Fuller, Sacramento, CA (US); Deborah Lynne Lysfjord, Gilbert, AZ (US); Sue Ann McConnell, Downers Grove, IL (US); Thomas Joseph Podsadecki, Chicago, IL (US); Scott Paul Smith, Oak Park, IL (US); David Russell Studenmund, Evanston, IL (US); Marlou Jean Campbell-Spry, Sugar Grove, IL (US); Edward Lewis Wristen, South Barrington, IL (US)

Correspondence Address: INDIANAPOLIS OFFICE 27879;BRINKS HOFER GILSON & LIONE, ONE INDIANA SQUARE, SUITE 1600, INDIANAPOLIS, IN, 46204-2033, US

(21) Appl. No.: 10/135,340

(22) Filed: Apr. 30, 2002

Related U.S. Application Data
(60) Provisional application No. 60/287,538, filed on Apr. 30, 2001.

(51) Int. Cl. G06Q 10/00; A61B 5/00

(52) U.S. Cl. 705/2; 600/300

(57) ABSTRACT

A system for outpatient treatment of patients diagnosed with a chronic health related condition includes a virtual office site operated with a server. The virtual office site is accessible over a network by a patient using a first browser, a physician using a second browser and a nurse case manager using a third browser. The patient and the patient's physician are provided secure communications to participate in a virtual office visit using the virtual office site. In addition, the physician may submit an electronic payment request with the virtual office site for reimbursement of services rendered. The nurse case manager is provided selective access to the communications to assist the patient in management of the patient's chronic health related condition.

This application claims the benefit pursuant to 35 U.S.C. §119(e) of Provisional U.S. Patent Application Ser. No. 60/287,538, filed on Apr. 30, 2001.

### BACKGROUND

1. Field of the Invention

This invention relates to outpatient treatment of chronic health related conditions and, more specifically, to a network based system for communication between a patient with a chronic health condition and the patient's physician.

2. Description of the Related Art

Chronic conditions such as asthma, diabetes, congestive heart failure and other similar diseases are prevalent in society today. Individuals with these conditions should typically engage in frequent communication with a physician to avoid episodes of acute illness. Physicians with patients having these chronic conditions typically spend significant amounts of time monitoring such patients and maintaining records. Frequent communication between these patients and their attending physicians requires regular office visits, telephone conversations or a combination of both. In some cases, frequent communication between patients and physicians does not occur due to the time it takes for patients to schedule appointments, attend appointments, wait to see the physician, etc. In these situations, patients may not see a physician often enough, may fail to perform adequate self-care and, as a result, may experience episodes of acute illness.

With the advent of on line communication via the Internet, other options in addition to these conventional communication techniques are now available. Relatively quick communication between a physician and a patient may now be provided through the use of written electronic communication such as, for example, e-mail. Another on-line communication technique involves the use of a message delivery service. Typically, the message delivery service operates to pass electronic messages generated by patients to their physicians and vice-versa. In general, messages may be transferred between a patient's computer and a physician's computer by the message delivery service.

One issue providing barriers to effective use of on-line communication is the reluctance of physicians to participate. The reluctance of physicians may be due to the concern of receiving voluminous and/or incoherent messages from patients. These messages must be deciphered and interpreted by the physician to ascertain the patient's condition. In addition, the process may be time consuming and not reimbursable to the physician. Further, record keeping regarding such on-line communication may also be a concern.

Another issue for patients with chronic conditions is the availability and effectiveness of the on-line communications. Patients with chronic health conditions maintain improved health with fewer episodes of acute illness requiring emergency care or hospitalization when frequent communication with their physician is available. To this end, monitoring by a third party to ensure the health of the patient does not deteriorate due to lack of contact with their physician has been found beneficial. With existing on-line communication systems, third party monitoring of on-line communications between the physician and the patient is unavailable. In addition, issues such as technical difficulties with using on-line communications, non-responsiveness of physicians/patients to a message and/or abuse of the privilege of on-line communication cannot be easily monitored to optimize effectiveness and availability.

### BRIEF SUMMARY

The present invention is defined by the following claims, and nothing in this section should be taken as a limitation on those claims. By way of introduction, the preferred embodiments described below include a method and system for outpatient treatment of a patient diagnosed with at least one chronic health condition. Outpatient treatment is performed with an outpatient treatment system that provides a virtual office site. The patient and the patient's physician communicate with a server over a network using a patient browser and a physician browser, respectively. The physician may conduct a virtual office visit with the patient when the patient initiates such a visit.

Initiation of the virtual office visit is performed through entry of data by the patient into a diagnosis template. The diagnosis template is specifically designed for the patient's chronic disease. The diagnosis template is interactively generated as a function of the patient's data entry to provide more concise and specific information to the physician. The patient may post the information contained in the diagnosis template on the virtual office site thereby initiating a virtual office visit. The physician is notified of the posting and may access the virtual office site to view a posted message containing the information from the diagnosis template. The physician may then respond to the patient by posting another message on the virtual office site that contains clinical information and advice for the patient. The physician may also elect to submit an electronic payment request to the virtual office site. The electronic payment request is automatically routed by the virtual office site to the patient's health benefits administrator for payment.

Prior to a patient and physician beginning communication with the outpatient treatment system, an approval process is undertaken. The approval process ensures both parties are willing and able to participate in disease management using the outpatient treatment system. The approval process may also be contingent upon the patient's willingness to participate in a care support program. The care support program includes a nurse case manager to assist in management of the chronic disease suffered by the patient. As part of this management, the nurse case manager may be provided access to the virtual office site to review communications between the patient and the patient's physician. The level of access provided to the nurse case manager is selectable by the patient.

The outpatient treatment system provides the physician and patient a communication interface for exchanging information to educate the patient as well as manage the patient's clinical condition and treatment plan. The use of the outpatient treatment system offers convenience and efficiency for both physicians and their patients with chronic illnesses. The virtual office site also provides relatively simple and efficient provisions for reimbursement of physicians for time spent conducting a virtual office visit. The patient, through more frequent and appropriate contact with his/her physician, may demonstrate mastery and compliance with the prescribed care plan. In addition, improved clinical oversight of the patient's health condition by the nurse case manager through the use of the outpatient treatment system may provide additional benefits to the patient.

### BRIEF DESCRIPTION OF SEVERAL VIEWS OF THE DRAWINGS

FIG. 1 is a block diagram of one embodiment of an outpatient treatment system for performing outpatient treatment of patients with chronic health conditions.

FIG. 2 is a more detailed block diagram of another embodiment of the outpatient treatment system illustrated in FIG. 1.

FIG. 3 is a flow diagram illustrating one embodiment of an approval process to qualify patients and the patients' physician to access the outpatient treatment system illustrated in FIG. 1.

FIG. 4 is a second part of the flow diagram illustrated in FIG. 3.

FIG. 5 is a third part of the flow diagram illustrated in FIG. 3.

FIG. 6 is one embodiment of a screen interface flow diagram for nurse case managers accessing the outpatient treatment system illustrated in FIG. 1.

FIG. 7 is a second part of the screen interface flow diagram illustrated in FIG. 4.

FIG. 8 is one embodiment of a screen interface flow diagram for patients accessing the outpatient treatment system illustrated in FIG. 1.

FIG. 9 is one embodiment of a screen interface flow diagram for physicians accessing the outpatient treatment system illustrated in FIG. 1.

FIG. 10 is one embodiment of a flow diagram illustrating operation of the outpatient treatment system illustrated in FIG. 1.

FIG. 11 is a second part of the flow diagram illustrated in FIG. 10.

FIG. 12 is a third part of the flow diagram illustrated in FIG. 10.

FIG. 13 is a fourth part of the flow diagram illustrated in FIG. 10.

### DETAILED DESCRIPTION OF THE PREFERRED EMBODIMENTS

The presently preferred embodiments provide an outpatient treatment system for secure communications between a patient with a chronic health condition and the patient's attending physician over a network. The system provides condition specific data entry templates for use by the patient to communicate with the physician. The data entry templates elicit concise and focused messages from the patient for the patient's physician. The outpatient treatment system also provides a direct and simple method for reimbursement of the physician. The physician may make requests for reimbursement directly to the patient's health benefits administrator using the outpatient treatment system. In addition, a nurse case manager is provided different levels of access to the outpatient treatment system, as selected by the patient. The nurse case manager assists in management of the patient's health care by acting as an advocate for the patient and to assist the patient in properly managing his/her chronic health condition(s).

The outpatient treatment system is a communication tool for improved self-management of chronic and/or high-risk health condition(s) suffered by a patient. The patient can use the system to improve communication with their attending physician and potentially avoid acute episodes of illness. Through participation of the patient and the patient's physician, identification of changes in the patient's condition may be detected at an early stage before an acute episode is unavoidable. The outpatient treatment system is not for appointment setting, general health inquiries or other more routine communications with a physician, nor is the system for emergency treatment or quick response. Rather, the system is an adjunct to care support for chronic and high-risk illness management within an established patient-physician relationship. In addition, the outpatient treatment system provides a nurse case manager the ability to be more involved and proactive in assisting patients with chronic condition(s) to further minimize episodes of acute illness.

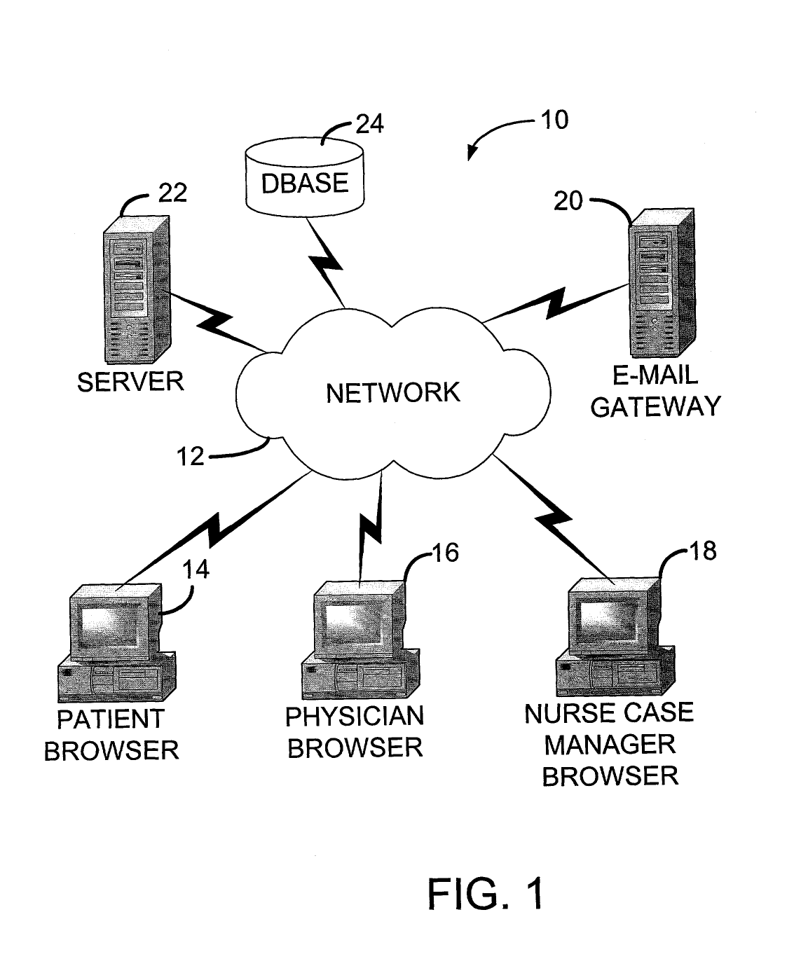

FIG. 1 illustrates one embodiment of an outpatient treatment system 10 providing a virtual office site in which a physician may conduct a virtual office visit. The outpatient treatment system 10 provides a virtual office site and includes a network 12, at least one first browser that is a patient browser 14, at least one second browser that is a physician browser 16, at least one third browser that is a nurse case manager browser 18, at least one email gateway 20, at least one server 22 and at least one database 24 communicatively coupled by the network 12 as illustrated. The number and configuration of the devices coupled with the network 12 are merely an illustrative example, and should not be construed as a limitation on the almost unlimited possibilities for configuration of outpatient treatment system 10. For example, the server 22 may be a plurality of servers connected by the network 12 to cooperatively perform various functionality related to the outpatient treatment system 10. As used herein, the term “connected” or “coupled” may mean electrically connected, optically coupled or any other form of coupling allowing the flow of data or some representation thereof between devices and components that are connected or coupled.

The network 12 may include the Internet, a public or private intranet, an extranet, and/or any other form of network configuration to enable transfer of data and commands. An exemplary network configuration uses the Transport Control Protocol/Internet Protocol (“TCP/IP”) network protocol suite, however, other Internet Protocol based networks are possible. Communications may also include IP tunneling protocols such as those that allow virtual private networks coupling multiple intranets or extranets together via the Internet. The network 12 may support application protocols, such as, for example, telnet, POP3, Mime, HTTP, HTTPS, PPP, TCP/IP, SMTP, proprietary protocols, or any other network protocols known in the art.

As used herein, the term Internet should be broadly construed to include any software application and hardware devices used to connect the browsers 14, 16 and 18, the server 22 and the email gateway 20 with an Internet service provider (not illustrated). The Internet service provider establishes the connection to the Internet. The browsers 14, 16 and 18, the server 22 and the email gateway 20 establish a connection to the Internet service provider using, for example, modems, cable modems, ISDN connections and devices, DSL connections and devices, fiber optic connections and devices, satellite connections and devices, wireless connections and devices, Bluetooth connections and devices or any other communication interface device.

As generally known in the art intranets and extranets are comprised of software applications and various computing devices (network cards, cables, hubs, routers, etc.) that are used to interconnect various computing devices and provide a communication path. The terms “intranet” and “extranet,” as used herein, should be broadly construed to include any and all hardware and software applications that allow the browsers 14, 16 and 18, the server 22, the email gateway 20 and any other computing devices to be communicatively coupled to share and transfer data and commands. Intranets and extranets are not limited to a particular physical location and may include multiple organizations using various communication protocols. As known in the art, various types of intranets and extranets exist and may be used with the presently preferred embodiments.

Communication within the network 12 may be performed with a communication medium that includes wireline based communication systems and/or wireless based communication systems. The communication medium may be for example, a communication channel, radio waves, microwave, wire transmissions, fiber optic transmissions, or any other communication medium capable of transmitting data in wireline and wireless based communication systems. During operation, the patient browser 14, the physician browser 16 and the nurse case manager browser 18 communicate with the server 22 and the email gateway 20.

The browsers 14, 16 and 18 may be any application running on a computer or other similar device capable of communicating over the network 12. The browsers 14, 16 and 18 include an Internet browser, proprietary software or any other application capable of forming a connection with the server 22 to send and receive information. In addition, the browsers 14, 16 and 18 include the ability to encrypt and decrypt data that is sent and received. Exemplary applications include Internet Explorer™ by Microsoft™ Corp. or Netscape Navigator™ by Netscape™ Corp. The browsers 14, 16 and 18 also include an e-mail application in communication with the email gateway 20. The e-mail application may be any application capable of sending and receiving e-mail messages. An exemplary e-mail application is Microsoft Outlook™ distributed by Microsoft™ Corp. The browsers 14, 16 and 18 may also include, for example, a server or any other devices and applications used to interface with and communicate via the network 12.

The email gateway 20 may be any computer, or similar device, that includes associated communications hardware and an application capable of handling incoming and outgoing electronic mail (e-mail) for the network 12. An exemplary embodiment is a computer that operates with Single Mail Transfer Protocol (SMTP) and Post Office Protocol 3 (POP3) using applications, such as, for example, Microsoft Windows NT™ and Microsoft Exchange Server™ by Microsoft™ Corp. The email gateway 20 may be multiple servers, a single server or may be part of the server 22 depending on the configuration and needs within the network 12.

The server 22 may be one or more computers or similar devices acting as an interface to the browsers 14, 16 and 18 for interactions with the applications or services available within the outpatient treatment system 10. The server 22 operates to authenticate the browsers 14, 16 and 18 and establish a secure connection between the browsers 14, 16 and 18 and the server 22. In addition, the server 22 allows applications the browsers 14, 16 and 18 are using to transparently access other resources within the outpatient treatment system 10.

The server 22 includes a firewall function, a network interfacing function and an application launching function. The firewall function provides protection against unauthorized access and controls traffic to the server 22. In the network interfacing function, the server 22 is responsible for interfacing with the network 12. In the application launching function, the server 22 may include applications to manage the logical flow of data and commands and keep track of the state of sessions. A session is a period of time in which the browsers 14, 16 and 18 are interacting with, and using the outpatient treatment system 10. In addition, the application launching function may include maintaining operative cooperation between multiple servers forming the server 22. Other applications operating within the server 22 may include encryption and decryption software. Exemplary encryption and decryption software encrypts commands transmitted across the network 12, and decrypts data received from the browsers 14, 16 and 18.

In the illustrated embodiment, the database 24 cooperatively operates with the server 22 as hereinafter described. In other embodiments, additional server computers and/or databases may be used. Where multiple servers and databases are used, applications within the servers coordinate operation of the outpatient treatment system 10 by enabling the databases to freely exchange data and other operationally related information by secure communication over the network 12. The server 22 also runs applications that store, maintain and allow interface to the data within the database 24. Applications, such as, for example, a database management system (DBMS) or other similar application may organize and coordinate the storage and retrieval of data from the database 24. The database 24 may be stored in a storage device, such as, for example, at least one hard drive, an optical storage media, or any other data storage device allowing read/write access to the data. The data within the database 24 may be stored in one centralized physical location or may be distributed among multiple physical locations (databases) within the network 12.

The database 24 of one embodiment contains data in one or more datafiles pertaining to each patient authorized to use the outpatient treatment system 10. In another embodiment, the database 24 may include multiple databases and the datafiles within the different databases may be linked to provide complete information pertaining to each patient. The databases providing complete information may include existing databases not specifically created for the outpatient treatment system 10. Such existing databases may include, for example, information related to patients and physicians, the patients health benefits administrator, the physicians provider network or any other information related to the patients and/or the physicians using the virtual office site. In one embodiment where the database 24 includes multiple databases and datafiles for each patient, information contained in the different database may be accessed over the network 12 by communication between the server 22 (servers) associated with the databases instead of being copied or otherwise duplicated. In yet another embodiment, where the database 24 includes multiple databases, the data within the databases 24 may be copied and provided as updates to the other databases. In still other embodiments, the data within the database 24 is encrypted for additional security.

Access to the database 24 over the network 12 allows the display and manipulation of data through selection of one or more of the datafiles. The level of access and manipulation of the database 24 is provided as a function of login information. The server 22 may perform verification based on the login information. Verification confirms that a requester has the authority to access and/or manipulate the datafiles being requested. In addition, verification allows penetration of the firewall. In other embodiments, the verification could be in the form of a password or any other technique for verifying authorization to access the database 24.

The server 22 of the presently preferred embodiments also operates at least one application that forms an interface for the browsers 14, 16, 18. The interface provides a virtual office site forming a communication path between the patients and the patients' physicians. In addition, the virtual office site provides access by the nurse case manager. The virtual office site is preferably implemented as an Internet, intranet or extranet accessible site. In this implementation, the virtual office site is a secure site requiring users to register or log in for access. In one embodiment, the virtual office site includes links to information within other systems on the network 12 pertaining to the physicians and the patients. Exemplary links include links to health benefits administrator systems for definitions of benefits available to patients, health claim administration systems for processing claims by patients and/or any other systems relating to administration of health care for the patients.

During operation of the previously discussed embodiments, a patient may access the server 22 over the network 12 using the patient browser 14. The patient logs on to the server 22 by, for example, entering a username and password. The logon includes verification of the patient as a user and provides access to only the datafiles in the database 24 pertaining to that patient. Patient information viewed with the patient browser 14 will be secured/encrypted from unauthorized viewing while traveling over the network 12.

Following successful login, the patient may enter the virtual office site. Within the virtual office site, the patient may view any messages posted by the patient's physician, as well as compose and post messages to the physician. In addition, messages related to the patient's chronic condition may be posted or links may be provided to other related information of interest to the patient. The reader should note that messages are posted at the virtual office site by applications operating the virtual office site. As used herein, the term “posted” refers to viewable information stored within the outpatient treatment system 10. Accordingly, information that has been posted is not sent as a message to any other location or device.

Initiation of a virtual office visit with the physician is initiated by the patient posting a message to the physician. Following initiation of the virtual office visit, multiple subsequent posted messages may be exchanged between the patient and physician regarding the virtual office visit. In one embodiment, the first message posted by the patient to initiate the virtual office visit is composed within a diagnosis template. As described later in detail, the diagnosis template is tailored specifically for the chronic disease suffered by the patient.

The physician similarly accesses the server 22 over the network 12 by logging on to the server 22 with the physician browser 16 using a password or other similar verification process. Following log in, the physician enters the virtual office site and is provided access to posted messages from the physician's patients and any other patient related information. The posted messages and other information accessible to the physician are encrypted for transmission over the network 12 to the physician browser 16. Access to the database 24 by the physician is limited to datafiles, messages and related information corresponding to the physician's login information. In addition, the physician may have access to other specific information such as, for example, care support guidelines, posted on the virtual office site.

The physician may read posted messages and post reply messages to patients. The posted reply messages may include a clinical response and other additional information for the patient. Posted messages relating to the subject of the office visit may continue to be exchanged by the patient and the physician until the patient's questions and/or concerns have been addressed. The posted messages relating to the virtual office visit remain accessible even after the visit is concluded. Additional requests and questions from the patient unrelated to the subject of the virtual office visit may be provided to the physician by initiating another virtual office visit.

In the presently preferred embodiments, the physician may direct the virtual office site to automatically prepare and submit an electronic payment request with the physician browser 16. The electronic payment request is prepared and submitted directly to, for example, the patient's health benefits administrator by the virtual office site. Submission of the electronic payment request may be with a secure communication link, encrypted email, datafile transfer from the database 24 or any other method of electronic data transmission. In other embodiments, the payment request may be submitted to, for example, the patient's bank account, a credit card account or any other payment service for the services provided to the patient. Selection of the automatic payment request is available to the physician within the virtual office visit initiated by the patient. Accordingly, the physician may request reimbursement for services rendered as part of responding to the patient's posted message.

In one embodiment, the virtual office site provides notification of posted messages with e-mail messages. In this embodiment, e-mail messages indicating new message postings are sent to the patient browser 14 or the physician browser 16 using the email gateway 20. Alternatively, e-mail messages may be sent to alphanumeric pagers, cellular telephones or any other device in communication with the server 22 or the network 12. In another embodiment, the e-mail message notifications sent to the patients and the physicians are also sent to a nurse case manager.

As previously discussed, the nurse case manager is not a physician's assistant. Rather, the nurse case manager assists patients in management of their chronic diseases. The nurse case manager may also access the server 22 with the nurse case manager browser 18. Logon to the server 22 may be accomplished by entry of a password or other similar verification. Access to the database 24 and other information relating to patients managed by the nurse case manager is provided through the virtual office site as a function of the login information. The nurse case manager uses the information to assist in management of the care of patients with chronic conditions. In another embodiment, the nurse case manager browser 18 is located on an intranet with the server 22. In this embodiment, an additional layer of security is added since the nurse case manager must first log on to the intranet, followed by logon to the virtual office site.

In the presently preferred embodiments, the virtual office site includes the capability for each of the patients to select the level of access provided to the nurse case manager. The level of access selection pertains to the nurse case manager's ability to access patient specific information. In one embodiment, the patient may chose whether the nurse case manager may access the content of messages posted by the patient and the patient's physician. In this embodiment, the access level may be changed by the patient at anytime using the patient browser 14.

In one embodiment, prior to providing patients and physicians access to the server 22, an approval process is implemented. The approval process ensures compatibility of the patients and the physicians with the operational aspects of the virtual office site. In addition, the approval process ensures the patient and the patient's physician both agree to use the outpatient treatment system 10 for additional communication. Finally, the approval process confirms that the patient is enrolled in, or is willing to enroll in, a care support program. Among other things, the care support program includes assignment of a nurse case manager to assist in the management of the patient's chronic disease(s).

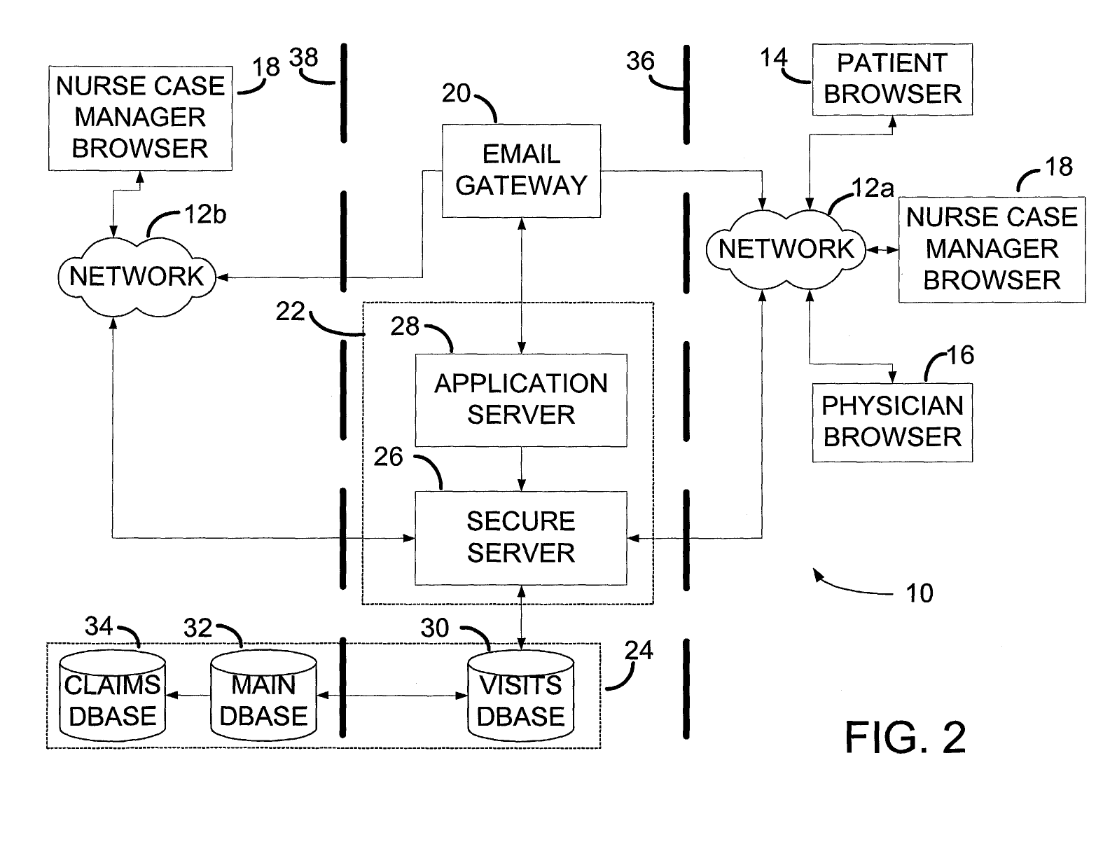

FIG. 2 is a more detailed block diagram of an exemplary embodiment of the outpatient treatment system 10 illustrated in FIG. 1. The outpatient treatment system 10 includes the patient browser 14, the physician browser 16, the nurse case manager browser 18, the email gateway 20, the server 22 and the database 24 communicating over the network 12 as in the previous embodiments. As further illustrated in FIG. 2, the server 22 of this embodiment includes a secure server 26 and an application server 28 communicating over the network 12. In addition, the database 24 includes a first database identified as a visits database 30, a second database identified as a main database 32 and a third database identified as a claims database 34 communicatively coupled as illustrated. In addition, the visits database 30 is preferably coupled with the secure server 26. Coupling within the database 24 and with the secure server 26 may include the network 12.

During operation of the illustrated embodiment, a patient may direct the patient browser 14 to communicate over a network 12a with the secure server 26 through a first firewall 36 to access the virtual office site as illustrated. In this embodiment, a portion of the network 12 is identified as “12a” to illustrate that the configuration of the network 12a preferably includes communication over the Internet or any other publicly accessible network. The patient browser 14 is provided a patient login page at the secure server 26. Following authorization of patient login information by the secure server 26, application(s) representing the patient interface functionality of the outpatient treatment system 10 may be served from the application server 28. Application(s) from the application server 28 may be served to the secure server 26, and may be presented to the patient browser 14 over the network 12a with a secure browser connection. The secure browser connection of one embodiment is a 128-bit secure socket layer (SSL) connection.

A physician may similarly direct the physician browser 16 to communicate over the network 12a with the secure server 26 through the first firewall 36 using the secure browser connection to access the virtual office site. Preferably, communication over the network 12a by the physician browser 16 similarly includes communication over the Internet. A physician login page may similarly be provided by the secure server 26. Once the login information of the physician has been authorized, application(s) on the application server 28 supporting physician use of the outpatient treatment system 10 may be served to the physician browser 16 over the network 12a via the secure server 26 with the secure browser connection.

A nurse case manager may operate the nurse case manager browser 18 to communicate over the network 12. The nurse case manager browser 18 may preferably communicate over the network 12a or over a network 12b based on the location of the nurse case manager. Communication over the network 12a is through the first firewall 36 similar to the patient browser 14. Following verification of login information entered with the nurse case manager browser 18 via a nurse case manager login screen, application(s) supporting the nurse case manager may be served from the application server 28 via the secure server 26 with the secure browser connection as previously described.

The portion of the network 12 is identified as “12b” to illustrate that the configuration of this portion of the network 12 preferably includes a network configuration not publicly accessible such as, for example, a wide area network (WAN), which does not include communication over the Internet. As known in the art, WANs are typical network configurations within business organizations. In one embodiment, the WAN is part of the business organization of the nurse case manager, such as, for example, a health benefits provider company.

The nurse case manager browser 18 may communicate over the network 12b through a second firewall 38 with the secure server 26. The secure server 26 may provide the nurse case manager login screen for entry of login information. Following successful login, the application server 28 may serve application(s) to the nurse case manager browser 18 over the network 12b via the secure server 26. It should be noted that since the network 12b of this embodiment is not publicly accessible, the secure browser connection is unnecessary.

One or more datafiles pertaining to each patient authorized to use the outpatient treatment system 10 may be stored in the visits database 30. Such datafiles may include, login information, activity within the outpatient treatment system 10, patient/physician linking, patient chronic disease(s) and any other information related to operation of the virtual office site by patients and physicians. The main database 32 may be an existing database that is linked to the visits database 30 through the second firewall 38 as illustrated. In these embodiments, both the main database 32 and the claims database 34 are part of the same business organization, such as, for example, a health benefits provider company. The main database 32 may include existing health and billing related information on patients. Existing health and billing related information may include, for example, the patient's name, social security number, unique ID, address, gender, date of birth, email address, phone number, insurance information, chronic disease diagnosis, employer, etc. In addition, where the patient is a dependent of an insured individual, similar information for the insured individual may also be included.

In one embodiment, existing information related to patients authorized to utilize the outpatient treatment system 10 may be transferred from the main database 32 and stored in the visits database 30. In other embodiments, the existing information may be dynamically retrieved from the main database 32 by the visits database 30 when needed. In addition, information in the visits database 30 may be similarly transferred to the main database 32 through the second firewall 38.

The claims database 34 is linked to the visits database 30 via the main database 32 as illustrated. In one embodiment, billing information from the visits database 30 may be transferred to the claims database 34 via the main database 32. Such billing information may be transferred as a datafeed to the claims database 34. In other embodiments, the claims database 34 may access the visits database 30 directly for the datafeed. Information provided in the datafeed may include, for example, the patient's identifying information, the physician's ID, a description of services performed by the physician and a charge for the services.

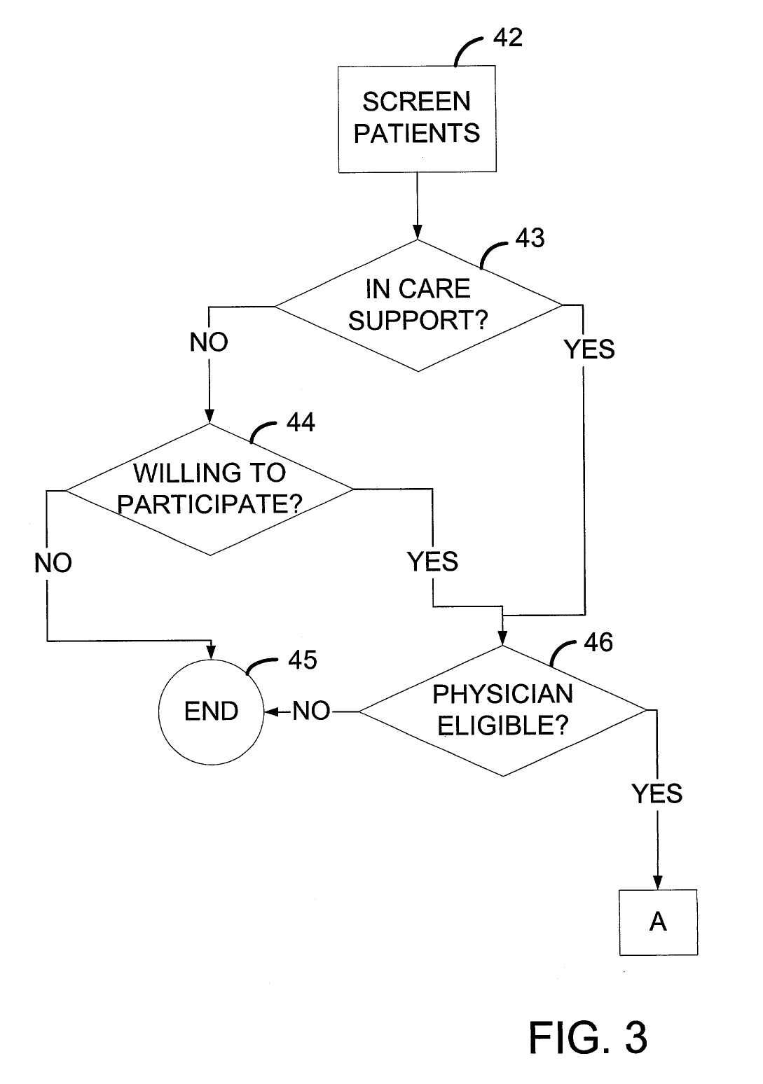

FIG. 3 is a block diagram illustrating one embodiment of the approval process that is hereinafter described with reference to FIG. 1. At block 42 screening for patients with chronic illnesses such as, for example, diabetes, asthma, congestive heart disease, human immunodeficiency virus (HIV), high-risk pregnancy, organ transplant, hypertension or any other chronic disease or illness is performed. At block 43, each of the patients with chronic conditions is checked for participation in a care support program involving a nurse case manager. If a patient is not participating, the patient is asked if they are willing to begin the program at block 44. If no, the approval process for that patient ends at block 45.

If the patient is already participating in the care support program, or the patient elects to participate, the patient's physician is checked for eligibility to use the outpatient treatment system 10 (FIG. 1) at block 46. Eligibility of the patient's physician involves, for example, availability of hardware configurable as the physician browser 16 (FIG. 1), physician's ability to participate in submission of electronic payment requests and any other criteria related to compatibility with the outpatient treatment system 10. If the physician is not eligible, the approval process ends at block 45.

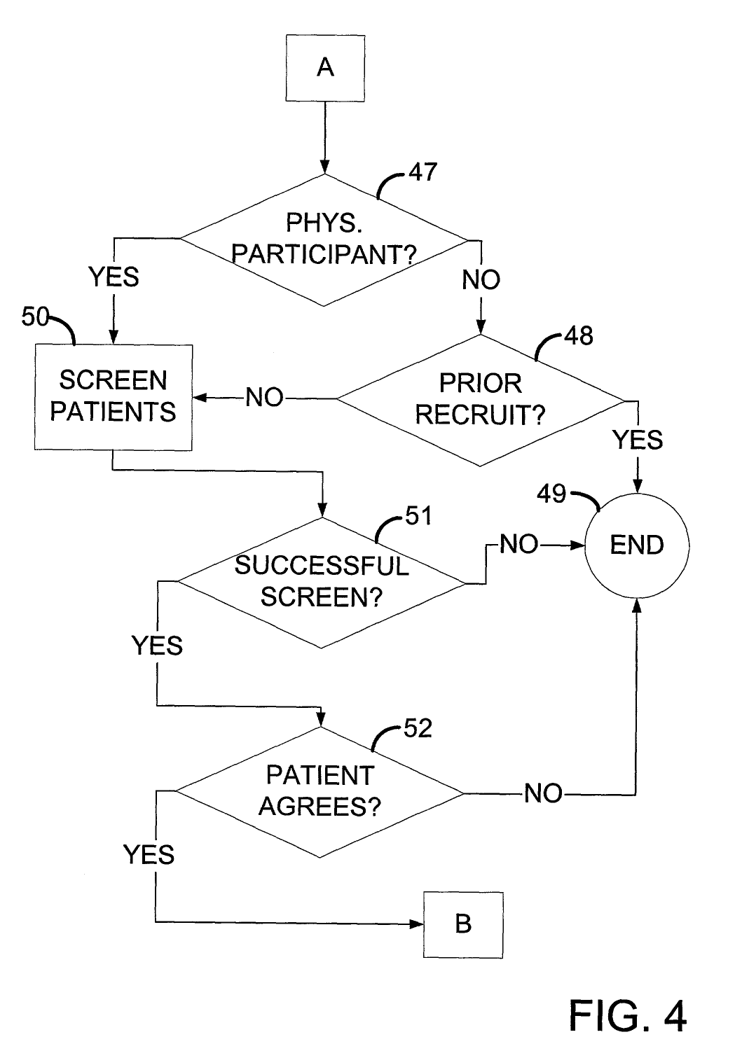

Referring now to FIG. 4, if the physician is eligible, determination of whether the physician has been successfully recruited to participate as a user of the outpatient treatment system 10 occurs at block 47. If no, at block 48, it is determined whether the physician was previously recruited for participation. If the physician was unsuccessfully recruited, the approval process for that patient ends at block 49.

If the physician was not previously recruited for participation, patients of that physician are screened at block 50. Screening may include an interview with each of the patients. During the interview, ascertainment of criteria, such as, for example, accessibility to hardware that is configurable to operate as the patient browser 14 (FIG. 1), the ability of the patient to understand and effectively communicate using the outpatient treatment system 10 and other similar criteria are explored. At block 51, the criteria are analyzed to determine whether the patient is a viable user of the outpatient treatment system 10. If no, the approval process ends at block 49. If the candidate is a viable user, the patient is asked if they desire to participate at block 52. If no, the approval process ends at block 49.

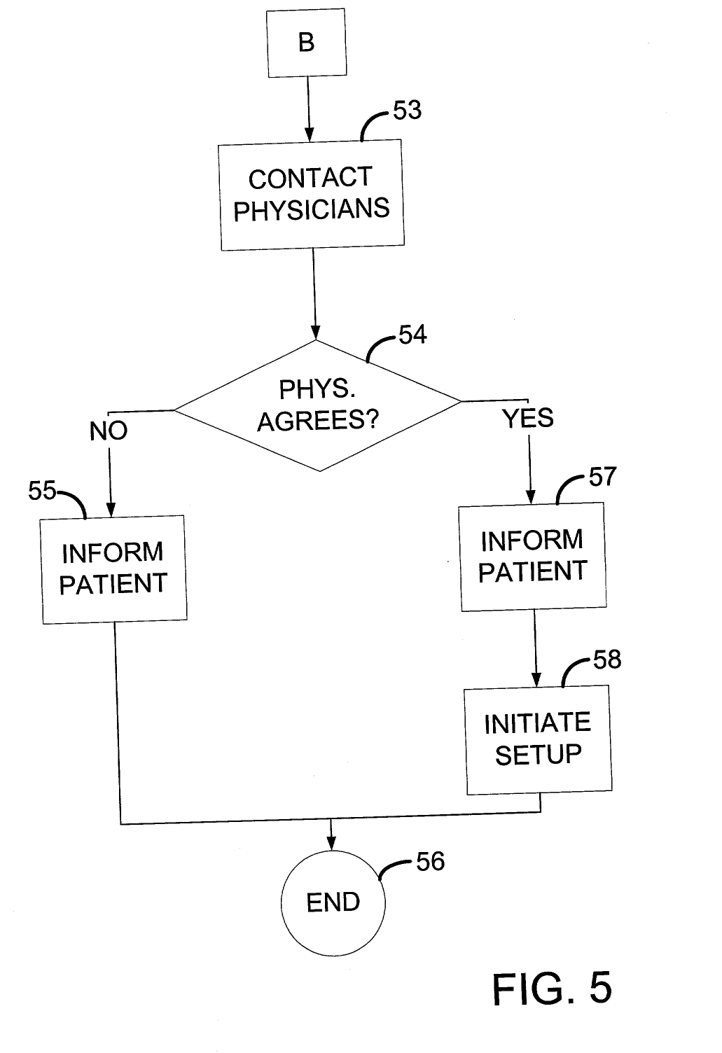

Referring now to FIG. 5, if the patient agrees to participate, the patient's attending physician is contacted at block 53. At block 54, the patient's attending physician is asked to evaluate the patient/physician relationship and determine if the outpatient treatment system 10 is a viable communication tool. If no, the patient is informed at block 55 of the physicians decline to participate and the approval process ends at block 56. If the physician agrees to participate with the patient, the patient is informed that they have successfully completed the approval process at block 57. At block 58, access by the patient and the physician are enabled to allow communication with the outpatient treatment system 10. The approval process ends at block 56.

Referring again to FIG. 1, enablement of the physician and the patient to access the outpatient treatment system 10 involves modification to the applications operating the virtual office site as well as changes to the database 24. An administrator of the server 22 such as, for example, the nurse case manager, may perform maintenance as well as enable access. In another embodiment, at least one administrator computer (not shown) may be included in the outpatient treatment system 10. The administrator computer is coupled with the network 12 and runs at least one client-server application providing access by an information system (IS) administrator to the server 22. Access by the IS administrator is for maintenance and modification of the virtual office site, the server 22 and the database 24. In yet another embodiment, a fourth browser that is at least one administrator browser (not shown) is included in the outpatient treatment system 10 to allow the IS administrator to maintain and perform modifications. In both embodiments involving the IS administrator, the nurse case manager, for example, performs functions related to managing the patients' chronic condition. The IS administrator, on the other hand, performs functions such as, for example, those related to hardware and application maintenance of the outpatient treatment system 10.

In one embodiment, the nurse case manager may enter patient information and perform other necessary modifications to provide a patient who has completed the approval process access to the virtual office site. In addition, the nurse case manager enters, on a patient-specific basis, the physician information required for the physician to use the virtual office site. Further, the nurse case manager links the patient and the physician to enable secure communications. Linking associates the patient with that patient's physician and identifies between whom access to private communication using the virtual office site is directed. In one embodiment, access by patients with multiple chronic diagnoses is configured for virtual office visits with more than one physician. In this embodiment, the patient will be linked with one physician for each diagnosis. The diagnosis and corresponding physician is selectable by the patient upon entry into the virtual office site.

Enabling access, maintenance and modification of the applications operating the virtual office site are performed by accessing the server 22 via the network 12. Alternatively, access to the server 22 may be performed at the site where the server 22 is located. In addition to maintenance, the nurse case manager may also access statistical information regarding virtual office site usage, view individual or multiple virtual office site visits and perform any other functions related to assisting in management of the health of patients with chronic conditions. The nurse case manager may also maintain, update and configure diagnosis specific intervention guidelines as will be later described. In another embodiment, enabling access, maintenance and modifications of the applications operating the virtual office site is performed by accessing multiple servers each containing different functionality and information pertaining to the virtual office site.

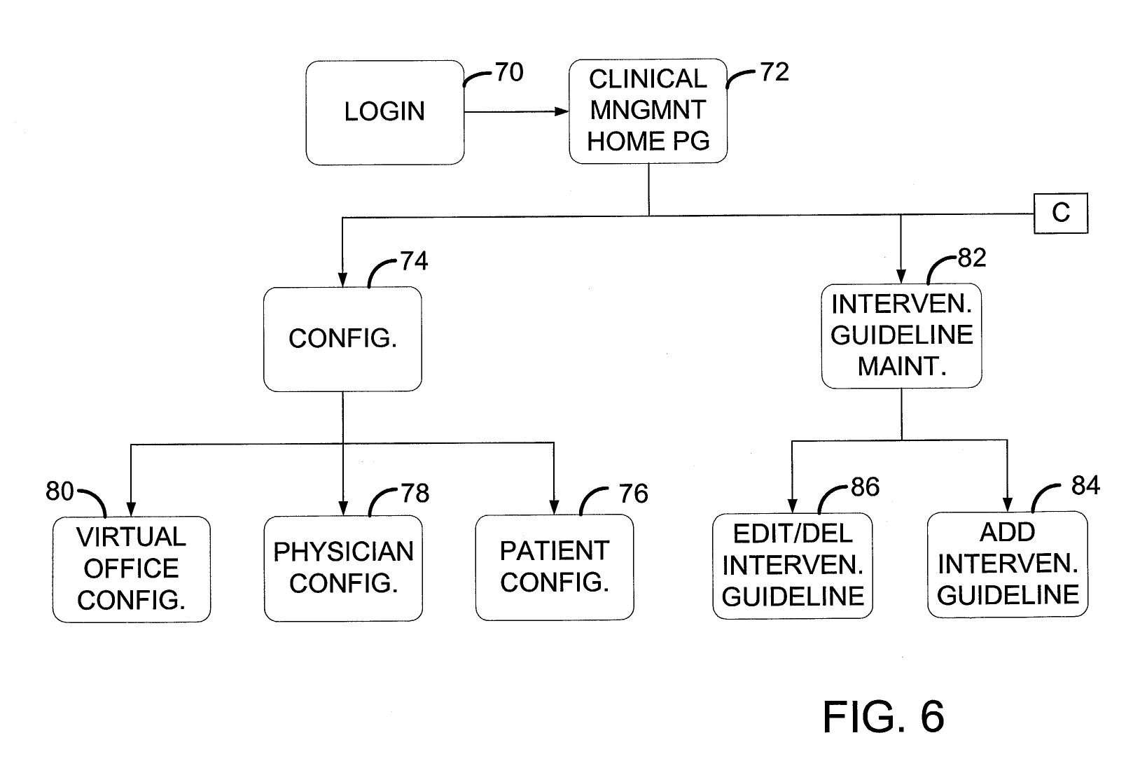

FIG. 6 is an interface screen flow diagram illustrating one embodiment of the functional interface of the nurse case manager to the virtual office site discussed hereinafter with reference to FIG. 1. The illustrated embodiment is but one implementation of the functional interface. In other embodiments, fewer or greater numbers of screens and selections may be used to access and manipulate information within the outpatient treatment system 10. In addition, as previously discussed, the functional interface and associated screens may be divided between those accessed by the nurse case manager and the IS administrator.

In one embodiment, upon accessing the virtual office site with the nurse case manager browser 18, the nurse case manager first encounters a login screen 70. Following successful login, a Clinical Management View Home Page 72 is provided. The Clinical Management View Home Page 72 includes a main menu allowing selection of information from the database 24. In the one embodiment, the main menu includes selections for Configuration, Intervention Guidelines Maintenance, Patient Information and Usage Reports. In other embodiments, fewer or more selection categories may be included pertaining to the virtual office site.

Selection of Configuration from the Clinical Management View Home Page 72 of one embodiment provides a Configuration Screen 74. The Configuration Screen 74 provides another menu for access to various functions performed to enable access by patients and physicians. In this embodiment, the menu selections include Patient Configuration, Physician Configuration and Virtual Office Configuration. In other embodiments, fewer or additional menu selections may be included. In still other embodiments, the setup functionality described may be provided by applications or other configuration techniques that do not include the Configuration Screen 74 and related screens, such as, for example, the client-server application(s) operated by the IS administrator.

In one embodiment, a Patient Configuration Screen 76 and a Physician Configuration Screen 78 are provided by selection of the Patient Configuration and Physician Configuration, respectively, from the menu. The Patient Configuration Screen 76 and a Physician Configuration Screen 78 include provisions to add physician and patient specific information to the database 24. The physician and patient specific information is used by the virtual office site to provide individually personalized data.

Exemplary information includes security clearance information such as user names, login identification, passwords, and other security related information. Other exemplary information includes diagnosed chronic conditions of the patient, patient allergies, other health conditions of the patient and any other information related to the patient. In addition, data utilized to customize applications in the server 22 for each of the patients and physicians may also be entered. Finally, physician/patient linking is configured to create a secure communication medium between the patient and the patient's physician. The physician/patient linking identifies the previously established relationship between the patient and the patient's physician and defines between whom the communications will occur within the virtual office site.

In one embodiment, the Patient Configuration Screen 76 also includes the ability to select the diagnosis template(s) for the patient. As previously described, the diagnosis templates are designed for a specific chronic condition suffered by a patient. When the patient wishes to initiate a virtual office visit, entry of information for the first posted message is in the form of a template based on the diagnosed chronic condition of that patient. Diagnosis templates may be selected for any chronic condition such as, for example, diabetes, asthma, congestive heart disease, HIV, high-risk pregnancy, organ transplant, hypertension or any other chronic health conditions. Patients with multiple diagnoses may be configured with a selection screen to select the diagnosis pertaining to the virtual office visit being initiated. In another embodiment, functionality to modify or create new diagnostic templates is also provided.

The diagnosis template of one embodiment includes a first screen with provisions for entry of a brief subject of the visit. In addition, the patient is presented with a list of symptoms. The list of symptoms provides chronic condition specific information as well as general information pertinent to the chronic condition. Consideration of the list of symptoms guides the patient to provide information to the physician typically needed to conduct a virtual office visit. The patient may identify which, if any, of the symptoms listed are currently being experienced. The symptoms selected interactively determine the content of a second screen of the template.

Within the second screen of the template, the patient may elaborate on the purpose of the visit in a free text format. For each symptom selected by the patient on the first screen, a corresponding text box is displayed on the second screen. The patient is asked to describe the symptom, when the symptom occurred and what the patient did about it. The patient is provided the opportunity to supply additional information through the template by answering additional questions. The answers to these questions may be entered in free form text fields. Information entered into the diagnosis template is formatted into a message and posted for the patient's physician when the patient elects to post the message.

Configuration of the diagnosis templates vary by diagnosis as well as by patient. Those patients with multiple diagnoses may be provided multiple diagnosis templates to select from. In another embodiment, diagnosis templates for the interaction of multiple diagnoses may also be provided. In yet another embodiment, diagnosis templates more generic in nature may be developed for wider ranges of diseases. In still other embodiments, the diagnosis template may include audio instructions, educational instruction or any other kind of communicative interaction responsive to the needs of the patient using the template. In still another embodiment, the templates provide iterative interaction with the patient. In this embodiment, subsequent questions are selected for inclusion in the template as a function of the patient's responses to previous questions.

[image]

[image]

[image]

[image]

[image]

Referring again to FIG. 6, selection of Virtual Office Configuration from the Configuration Screen 74 provides a Virtual Office Configuration Screen 80. The Virtual Office Configuration Screen 80 includes capability to customize the virtual office site for each physician/patient relationship. Exemplary customization includes links for the patient to information pertaining to the chronic conditions suffered by that patient, links for the physician to the patients electronic health records, links for the electronic payment request submission by the physician and any other content within the virtual office visit specific to the physician or the patient. For example, configuration of a notification system for the previously described e-mail notification of posted messages is configured from the within the Virtual Office Configuration Screen 80.

One embodiment of the virtual office site includes an intervention guideline system. The intervention guideline system is provided as a monitoring mechanism. The monitoring mechanism indicates when predetermined thresholds are reached for pre-specified variables pertaining to the patients' chronic condition(s). The indications may be configured by the nurse case manager as intervention guidelines. In one embodiment, notification that predetermined thresholds have been reached for intervention guidelines may be indicated by e-mail messages generated by applications within the virtual office site. The e-mail messages indicate the time, date, the intervention guideline and the condition that generated the notification. The e-mail messages may be directed to, for example, the nurse case manager. In other embodiments, e-mail messages may be directed to any other individual or organization responsive to the particular intervention guideline for which a message is generated.

In the presently preferred embodiment, when the Intervention Guidelines Maintenance is selected from the Clinical Management Home Page 72 an Intervention Guideline Maintenance Screen 82 is provided as illustrated. The Intervention Guideline Maintenance Screen 82 provides for maintenance, management and update of intervention guidelines applied to all patients diagnosed with a particular chronic condition. A list of intervention guidelines currently implemented to address the needs of patients diagnosed with a particular chronic condition are provided on the Intervention Guideline Maintenance Screen 82. The list includes the diagnosis for the corresponding guideline, a variable being monitored, the threshold for the corresponding variable and the location the e-mail notification will be directed to. In another embodiment, intervention guidelines may be implemented for each patient individually based on the individuals chronic condition(s) or other patient specific factors.

From within the Intervention Guideline Maintenance Screen 82 selection of an Add Intervention Guideline Screen 84 is available. The Add Intervention Guideline Screen 84 provides a template allowing the implementation of intervention guidelines. In one embodiment, selections are available in a drop down box within the Add Intervention Guideline Screen 84. The drop down box provides selection of diagnosis, such as, for example, diabetes, asthma, congestive heart disease or any other diagnosis. In addition, one of plurality of variables included in the database 24 (FIG. 1) corresponding to the diagnosis may also be selected. Further, a threshold corresponding to the variable may also be selected. The Add Intervention Guideline Screen 84 also includes the ability to enter an e-mail address to which notifications will be sent when the threshold of the selected variable is exceeded for any patient with the selected diagnosis.

Exemplary intervention guidelines that may be configured within the Add Intervention Guideline Screen 84 include excessive usage of the virtual office visits, messages posted for extended periods without being read, undeliverable e-mails and limited remaining virtual office visits. Excessive usage notification may be based on monitoring the number of virtual office visits initiated with the threshold set to a predetermined number, such as, for example, three virtual office visits initiated in a one-week period. Extended period message posting may be performed by monitoring for unread messages that have been posted for more than a predetermined period, such as, for example, 72 hours. E-mail delivery failures may be generated, for example, when e-mail notification of posted messages fails. Where a predetermined number of virtual office visits are available, notification may be issued when, for example, the patient has initiates each of the last two visits available. In other embodiments, other notifications could be issued such as, for example, excessive number of messages in a virtual office visit, multiple unsuccessful attempts to operate some aspect of the virtual office site, or any other monitored parameters indicative of non-routine operation by the patient or the physician.

Currently implemented intervention guidelines may also be edited. In one embodiment, selection of a currently implemented intervention guideline from the list in the Intervention Guideline Screen 82 provides an Edit/Delete Intervention Guideline Maintenance Screen 86. Within the Edit/Delete Intervention Guideline Maintenance Screen 86, the selected intervention guideline may be edited.

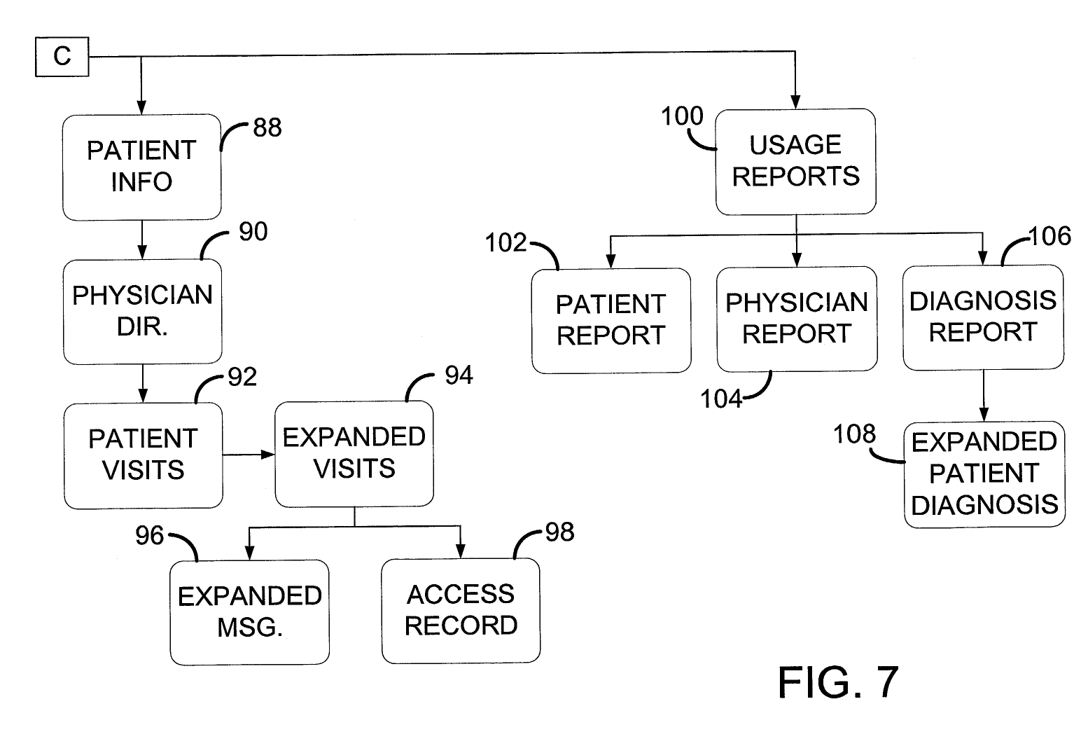

Referring now to FIG. 7, upon selection of Patient Information from the menu on the Clinical Management Home Page 72 (FIG. 6), a Patient Information Screen 88 is provided. The Patient Information Screen 88 includes an alphabetical list of all patients who have been enabled to access the virtual office site. In one embodiment, the patient's name appears as a hyperlink on the Patient Information Screen 88. Selection of the patient's name will provide a Physician Directory Screen 90 for that patient.

The Physician Directory Screen 90 of one embodiment displays the names of all physicians either currently or previously linked with the patient for communication using the virtual office site. In addition, the chronic condition for which the physician is treating the patient is displayed. The Physician Directory Screen 90 also includes indication of status of the physician/patient relationship as active or inactive. A status indication of inactive indicates the physician and patient are not currently enabled to participate in a virtual office visit. Active status indications of one embodiment also include an indicator to identify in progress virtual office visits containing posted messages that have not been read by the intended recipient (e.g. either the physician or patient). In one embodiment, the Physician Directory Screen 90 may also include a selection for a Patient Visit Screen 92 for each physician listed. The selection of the Patient Visits Screen 92 is available only for those physicians who have had at least one patient initiated virtual office visit.

The Patient Visits Screen 92 allows the nurse case manager some level of access to the posted messages between the patient and the patient's physician. The level of access is selected by the patient and determines whether the nurse case manager is permitted to view the contents of the posted messages or simply view the list of posted messages. In another embodiment, the patient can select individual posted messages as viewable or non-viewable by the nurse case manager.

When posted messages are viewable by the nurse case manager, the Patient Visits Screen 92 displays the virtual office visits for a selected physician in a summary view. In one embodiment, each of the virtual office visits is represented in summary form in a corresponding visit header. The visit headers include the name of the patient, the name of the physician, the date of the initiation of the virtual office visit, the date of the last message posted pertaining to the visit, the subject of the visit and/or any other criteria related to summarizing the visit. In another embodiment, the information in the visit headers is selectable as a sort mechanism for the summary list of virtual office visits displayed. Also included in each of the visit headers is indication of whether the physician has submitted an electronic payment request for that virtual office visit. Similar to the Physician Directory Screen 90, each visit header also includes indication of unread messages posted for the corresponding virtual office visit.

In one embodiment, each visit header also includes the ability to select an Expanded Visits Screen 94. The Expanded Visits Screen 94 provides a detailed view of the virtual office visit displayed in the summary view of the Patients Visits Screen 92. In one embodiment, expansion of the summary view is performed by selection of an expansion icon displayed within the visit header. In another embodiment, expansion of the summary view may be performed by selection of the information displayed within the visit header.

The Expanded Visits Screen 94 of one embodiment includes a message header for each message posted by the patient or the physician for the selected virtual office visit. Each of the message headers includes the author's name and the date/time stamp of when the message was posted. The message posted by the patient who initiated the virtual office visit may also be identified and includes the subject of the visit from the diagnosis template. As previously described, the diagnosis template provides a structure for the format and data content of the posted message initiating the virtual office visit. In one embodiment, posted messages from the patient and from the physician are differentiated by, for example, use of different text fonts, text colors, backgrounds or other similar contrasting notation. The Expanded Visits Screen 94 may be collapsed back to the Patient Visits Screen 92 by selection of the expansion icon and/or information displayed in the visit header.

Posted messages listed in the Expanded Visits Screen 94 include read and unread indication that may be, for example, a separate indicator, color changes, textual changes or any other form of visual differentiation. In one embodiment, time zone indication is also provided. Similar to the visit header, the message header also includes an expansion icon allowing messages within the Expanded Visits Screen 94 to be expanded and collapsed with the icon, or by selecting the message header. In one embodiment, when one or more of the message headers is selected for expansion, an Expanded Message Screen 96 is provided. The Expanded Message Screen 96 displays the expanded message(s) as well as any other message headers that remain unexpanded.

An Access Record Screen 98 is also available from the Expanded Visits Screen 94. The Access Record Screen 98 of one embodiment provides a history of users who have accessed the posted messages listed in the Expanded Visits Screen 94. The history provides each instance when a posted message was accessed and includes a date/time stamp of the access and the identity of the accessing user. For example, the Access Record Screen 98 indicates the date and time when individual messages were accessed by the physician, the patient or the nurse case manager based on login information.

In the presently preferred embodiments, when the patient has selected that the nurse case manager not view the contents of posted messages, the Patient Listing Screen 88, The Physician Directory Screen 90 and the Access Record Screen 88 remain the same as previously described. However, the Patient Visits Screen 92 and the Expanded Visits Screen 94 are modified and the Expanded Messages Screen 96 is unavailable. In one embodiment, modification of the Patient Visits Screen 92 and the Expanded Visits Screen 94 includes removal of the subject of the virtual office visit and any other patient specific information related to summarizing the visit.

As further illustrated in FIG. 7, selection of Usage Reports from the Clinical Management View Home Page 72 (FIG. 6) provides a Usage Reports Screen 100. The Usage Reports Screen 100 provides general information about the outpatient treatment system 10 (FIG. 1). The information may be viewed on screen or selected for printing in hardcopy form to a printer or other similar device electrically connected with the nurse case manager browser 18 (FIG. 1) or the network 12 (FIG. 1).

In one embodiment, the information provided on the Usage Report Screen 100 includes general usage statistics for the virtual office site. In addition, a date range input field is provided. The date range input field allows entry of a range for any data within the database 24 (FIG. 1). As a function of the date range selected, one of a plurality of report types may be selected. In the presently preferred embodiment, reports available for selection include a Report by Patient, a Report by Physician and a Report by Diagnosis. In other embodiments, any number of other reports or variations of the preferred reports is possible.

Selection of the Report by Patient provides a Patient Report Screen 102. The Patient Report Screen 102 provides listings of all patients participating in dialogue with physicians using the virtual office site during the specified date range. The report may indicate, for example, total number of patients participating in a pre-specified period of time, the total number of visits per patient or any other patient related information. In another embodiment, patients may be categorized by employer, health benefits administrator etc. In this embodiment, reports may indicate for example, total number of patients of a specified employer engaging in virtual office visits during a pre-specified period of time, the total number of patient visits for a specified employer or the number of patients participating for a specified employer.

The Report by Physician selection similarly provides a Physician Report Screen 104. In one embodiment, the Physician Report Screen 104 includes a listing of all physicians actively participating in dialogue with patients using the virtual office site. In this embodiment, indication may include, for example, the total number of patients per physician, the total number of virtual office visits for each physician in a predetermined time period or any other information related to interaction of the physicians with the patients.

A Diagnosis Report Screen 106 is provided upon selection of Report by Diagnosis from the Report Usage Screen 100. The Diagnosis Report Screen 106 of one embodiment is a summary screen indicating, for example, the total number of active patients per diagnosis and/or the total number of visits for a selected time period as well as any other diagnosis related information. Within one embodiment of the Diagnosis Report Screen 106 is provided selection of an Expanded Diagnosis Report Screen 108. The Expand Diagnosis Report Screen may indicate, for example, the total number of patients and total number of virtual office visits for a particular diagnosis.

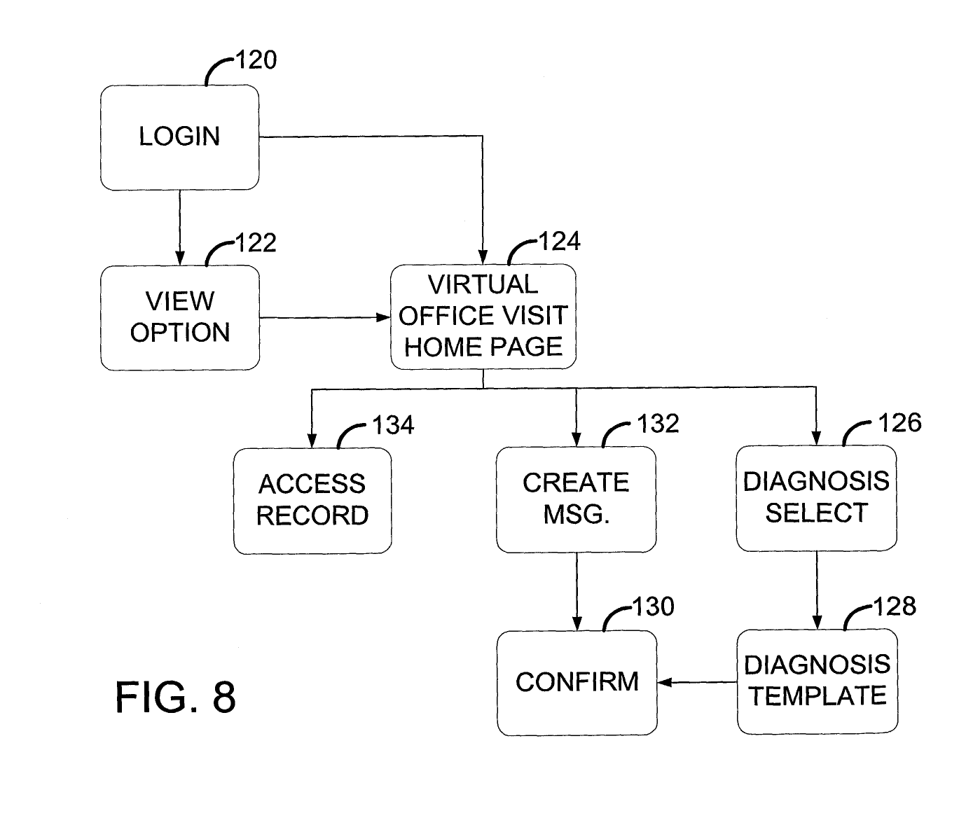

FIG. 8 illustrates one embodiment of a screen flow diagram of the virtual office site interface for patients. Patients may enter the virtual office site with access to view and create virtual office visits and their associated messages. When accessing the virtual office site with the patient browser 14 (FIG. 1), a Login Screen 120 is initially encountered. For patients who have completed the approval process, upon entry of a valid username and password in the Login Screen 120, access is provided If the patient is a first time visitor to the virtual office site, a Nurse case manager View Option Screen 122 is provided. The Nurse case manager View Option Screen 122 includes a dialog box requesting the patient to choose the level of access of the nurse case manager. In the presently preferred embodiment, access is a yes/no option providing the nurse case manager access to the contents of the posted messages or not. The Nurse case manager View Option Screen 122 is only provided at the initial login of a patient. Once the access level has been set, the patient may change the selection at any time. Conversely, if this is not the first time the patient has entered virtual office site, a Virtual Office Visit Home Page 124 will be displayed following login.

In one embodiment, if a valid username and password are entered and the database 24 (FIG. 1) does not include valid linking to the patient's physician, one of two messages is displayed. In those cases where a patient/physician link does not exist for the patient, an error message indicates there is currently no physician with which to conduct virtual office visits. If the patient had a prior link to a physician that is no longer active (e.g. the physician or the patient opted out of virtual office visits), this is indicated and the patient is limited to viewing past virtual office visits and related posted messages. In either case, the ability to create and post a message to create a new virtual office visit, or continue an existing visit, is not available.

The Virtual Office Visit Home Page 124 is the main screen for the patients accessing the virtual office site. As a function of the patient's login information, a list of the patient's previous virtual office visits is displayed. If the patient has multiple chronic conditions and therefore can conduct virtual office visits with more than one physician, any virtual office visits previously initiated with each physician are displayed. In one embodiment, each of the virtual office visits are displayed in the visit header as previously described and include indication of any unread messages. As in the previously described embodiments, the visit header may be expanded and collapsed with the expansion icon or by selecting the information within the visit header. Expansion of the visit header provides display of the message header that may be further expanded to display the individual messages as previously described.

The ability to initiate a new virtual office visit is also included in the presently preferred Virtual Office Visit Home Page 124. Initiation of a new virtual office visit provides access to the diagnosis template(s) as previously described. In one embodiment, where the patient has multiple chronic conditions, a Diagnosis Selection Screen 126 is provided. The Diagnosis Selection Screen 126 includes selections for each chronic condition suffered by that patient. Selection of one of the chronic conditions provides a Diagnosis Template Screen 128 corresponding to the selected chronic condition. In one embodiment, only one physician may be associated with each diagnosis, so the diagnosis selection will dictate the appropriate physician to post the message for. In another embodiment, multiple physicians may be listed and the patient selects the physician with which a virtual office visit is desired. In yet another embodiment, the patient is diagnosed with a single chronic condition. In this embodiment, initiation of a new virtual office visit provides the Diagnosis Template Screen 128 corresponding to the patient's condition.

Included in the Diagnosis Template Screen 128 is the ability to post the information when data entry by the patient into the diagnosis template is complete. Upon posting, a new virtual office visit is initiated. The information within the diagnosis template is formatted to a text message and posted in the virtual office site for retrieval by the patients physician. In addition, the patient's physician is sent notification that a message has been posted. Following submission, a Confirmation Screen 130 is provided. The Confirmation Screen 130 indicates that the patient's message has been posted. In addition, the Confirmation Screen 130 provides selections for further functions, such as, for example, logoff the virtual office site, return to the Virtual Office Visit Home Page 124 or any other functionality available to the patient.

The Virtual Office Visit Home Page 124 also includes a create message selection. The create message selection is available within an existing virtual office visit. When the create message selection is chosen, the patient is provided a Create Message Screen 132. The Create Message Screen 132 of one embodiment includes a form allowing free form text entry of a message. In another embodiment, a diagnosis template is provided. The diagnosis template may be generated as a function of a message from the patient's physician, or may be selected by the physician. Included in the Create Message Screen 132 is the capability to post a message. Messages are posted by the virtual office site for retrieval by the patient's physician. Posting of the message generates notification of the physician and provides the previously described Confirmation Screen 130.

In one embodiment, the Virtual Office Visit Home Page 124 also includes selection of an Access Record Screen 134. The Access Record Screen 134 is similar to the previously described Access Record Screen 98 (FIG. 7) and provides the ability to view a history of who has accessed messages generated by the patient. In other embodiments, additional functionality may be accessible from the Virtual Office Home Page 124. Exemplary functions include, for example, useful links to self care information pertaining to the chronic condition(s) suffered by the patient, contact information for the patient's physician, emergency services contact information, terms of use, help screens or any other functionality pertinent to the patient's condition or navigation of the virtual office site.

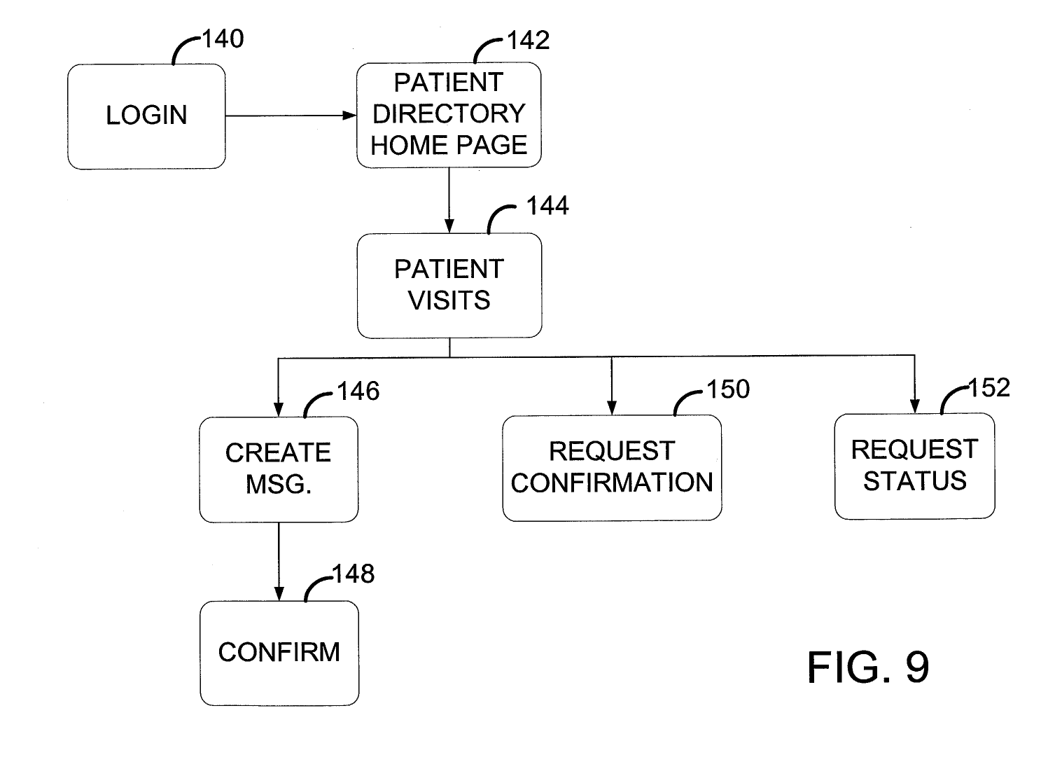

FIG. 9 illustrates one embodiment of an interface screen flow diagram depicting the functional interface for physicians accessing the virtual office site. The physician is provided access to view and post messages in reply to messages posted by the physician's patients. In addition, the physician is provided the ability to submit electronic payment requests for services rendered in connection with the virtual office visits. Upon accessing the server 22 (FIG. 1) with the physician browser 16 (FIG. 1), the physician is provided a Login Screen 140. In one embodiment, if valid login information is entered and a physician/patient linking does not exist, the physician receives an error indicating no patients are currently available with which to conduct virtual office visits. If the physician has active relationships with patients using the virtual office site, the physician is provided a Patient Directory Screen Home Page 142.

The Patient Directory Screen Home Page 142 displays the names of all patients either currently or previously communicating with the physician using the virtual office site as a function of the login information. One embodiment includes indication of unread posted messages from each patient as well as the status of the physician/patient relationship as actively or inactively linked. The Patient Directory Screen Home Page 142 also provides selection of a Patient Visits Screen 144 for those patients who have initiated at least one virtual office visit with the physician.

In one embodiment, the Patient Visits Screen 144 includes a list of the virtual office visits for each patient in the form of the previously described visits header. The visits header will include indication of whether any unread posted messages are present as well as whether the physician has previously submitted an electronic payment request for the virtual office visit through the virtual office site. As in the previous embodiments, each of the visits headers may be expanded to provide the message headers for the virtual office visit. In addition, the message headers may be further expanded to provide the individual posted messages. From within the visit headers or the message headers, the physician is provided a new message selection and a submit electronic payment request selection.

The new message selection provides a Create Message Screen 146. In one embodiment, the Create Message Screen 146 includes the ability to type free form text and post the message. In another embodiment, the Create Message Screen 146 includes the ability to end the virtual office visit. When the physician elects to end the virtual office visit, indication that the virtual office visit is completed is provided in the message posted by the physician. In addition, the virtual office site eliminates the ability of the patient to post further messages with regard to that virtual office visit.

Messages are posted for the patient associated with the virtual office visit. When a message is posted, the message is added to the list of messages previously posted within the virtual office visit. In addition, notification is provided to the patient and a Confirmation Screen 148 is provided. The Confirmation Screen 148 is similar to the Confirmation Screen 130 previously described with reference to FIG. 8.

In one embodiment, the submit electronic payment request selection is available for any patient visit that has been expanded and not already billed. When the submit electronic payment request selection is chosen by the physician, an electronic request for payment will be automatically submitted to, for example, the patient's health benefits administrator by the virtual office site. The mechanisms for submission of the request will vary depending on the party making payments. Setup of the submission mechanism is part of the configuration of the virtual office site as previously described. The submission mechanism is deliberately simple for the physician to provide a convenient way to request payment for services rendered.

In one embodiment, once the submit electronic payment request selection is chosen, the physician will be presented with a dialog box asking for confirmation of submission of an electronic request for payment. If confirmed, the physician will be presented with a Request Confirmation Screen 150. The Request Confirmation Screen provides patient specific information, the party who the request is directed to, the date and time and any other information pertinent to the transaction.

In one embodiment, the physician is able to track the “status” of the request through a Request Status Screen 152. The Request Status Screen 152 provides a tracking mechanism indicating who is currently in receipt of the request, the payment status of the request and any other information related to tracking.

Once the physician has submitted a payment request for a virtual office visit, the submit electronic payment selection is no longer available from the Patient Visits Screen 144. Instead, indication that a request has been submitted is displayed and selection of the Request Status Screen 152 is provided. The physician may, at any time, select the Request Status Screen 152 to review information relating to submission of a request for payment for a virtual office visit.

The Patient Directory Home Page 142 may also include other functionality for the physician. Exemplary functionality includes, for example, links to sites related to chronic conditions treated by the physician, guidelines dictated by the patient's health benefits administrator, electronic health records, prescription drug services and any other functionality related to providing health care to the patients and navigating within the virtual office site. In addition such information as terms of use of the virtual office site, common procedures and information related to chronic conditions and/or any other information may be accessible from the Patient Directory Home Page 142.

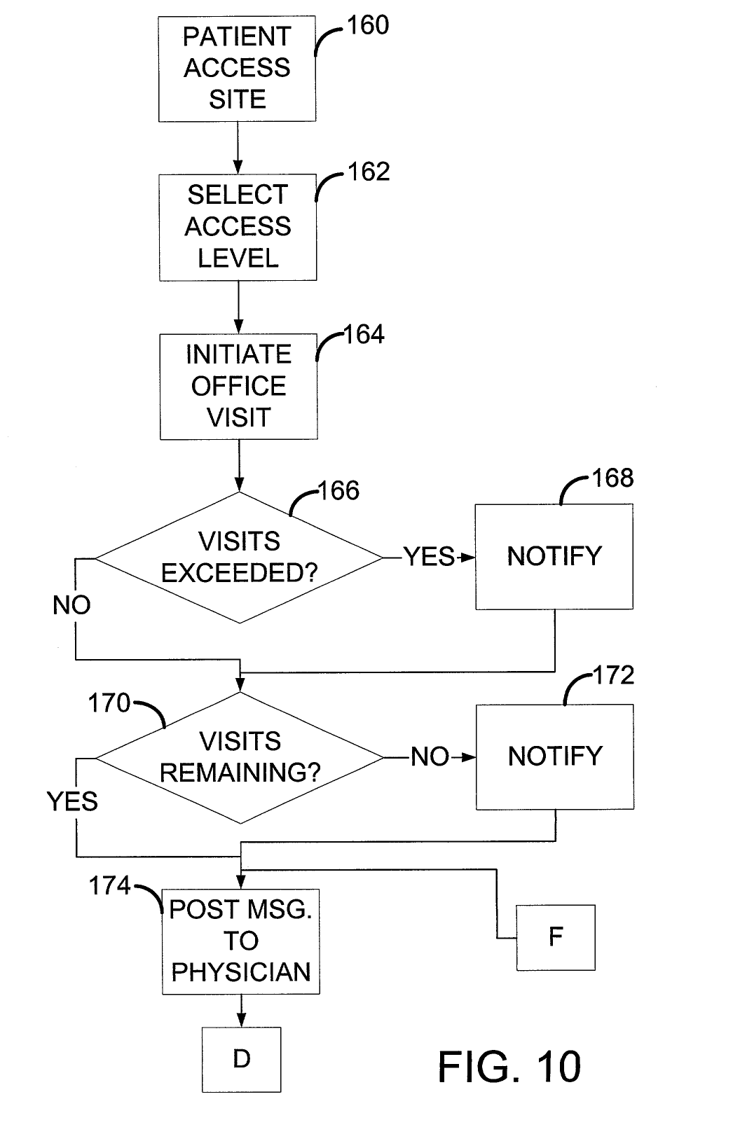

FIG. 10 is a flow diagram illustrating operation of one embodiment of the outpatient treatment system 10 with reference to FIGS. 1-9. Following the approval process and configuration by the nurse case manager, the patient and the patient's physician are enabled to access the virtual office site. At block 160, the patient accesses the virtual office site with the patient browser 14. Following successful login, the patient may access information related to the patient's chronic condition. At block 162, the patient selects the level of access for the nurse case manager. The patient initiates a virtual office visit by posting information pertaining to the patient's chronic condition in the form of a diagnosis template and then logs off at block 164.

At block 166, the intervention guidelines are used to check the database 24 to ensure the allowable number of virtual office visits within a predetermined time period have not been exceeded. If yes, notification is generated and sent to the nurse case manager at block 168. Where the patient allows access, the nurse case manager reviews the content of the messages and determines whether to call the patient to discuss. Conversely, where the patient has not allowed access, the nurse case manager calls the patient to discuss. Following notification of the nurse case manager, or confirmation that the allowable number of visits is not exceeded, the intervention guidelines are used to check the database 24 to confirm the patient has greater than a threshold number of virtual office visits remaining at block 170. If less than the threshold number of visits remains, notification is again provided to the nurse case manager at block 172. Following notification, or confirmation that the remaining number of visits is greater than the threshold, the message is posted for the patient's physician at block 174.

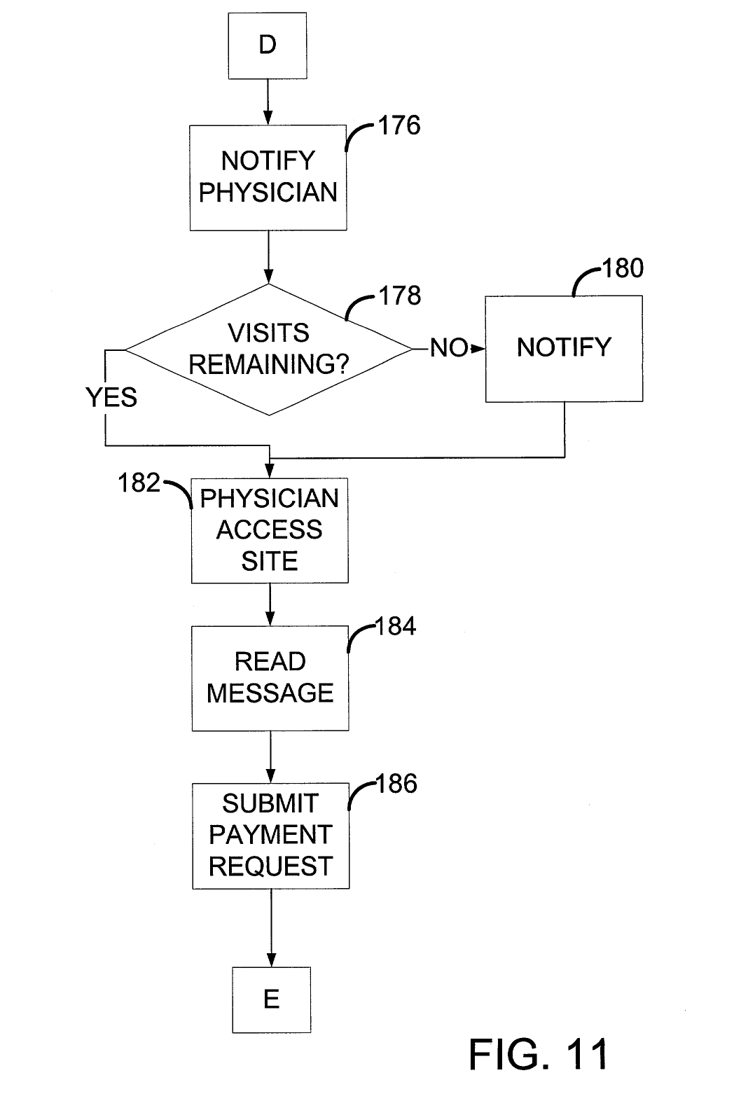

Referring now to FIG. 11, at block 176, the patient's physician is provided notification that a message has been posted. The intervention guidelines are used at block 178 to determine whether the posted message remains unread for longer than a predetermined period of time. If yes, the nurse case manager is notified at block 180. Once notified, where the patient has allowed access, the nurse case manager reviews the content of the posted message and determines whether contacting the physician is necessary. If the nurse case manager is not provided access to the content of the posted messages, the nurse case manager contacts the physician.

The physician logs on to the virtual office site with the physician browser 16 at block 182. At block 184, the physician selects the patient who originated the message and reads the posted message. At block 186, the physician composes a response to the patient's message and submits an electronic payment request.

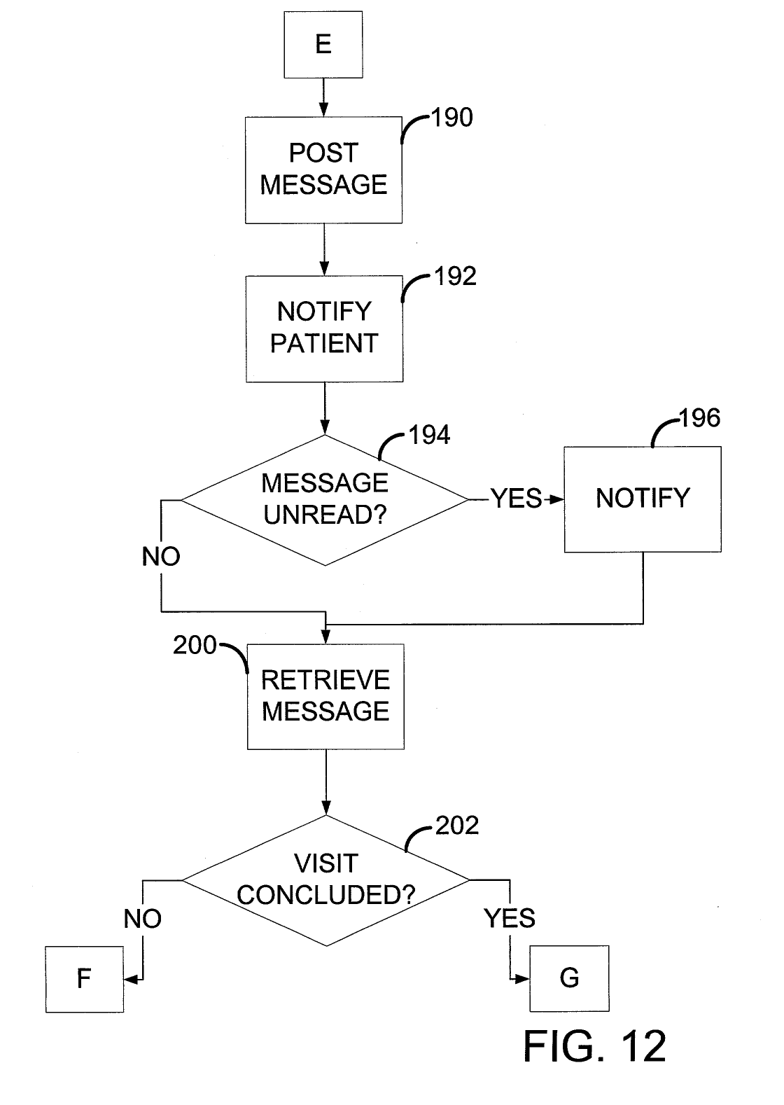

Referring now to FIG. 12, the physician posts the message at block 190 and logs off the virtual office site. At block 192, the patient is notified of a posted message. The intervention guidelines are again used at block 194 to determine if the message remains unread for longer than a predetermined period of time. If yes, the nurse case manager is notified at block 196 and takes action as previously described. If the predetermine time has not expired, or following notification of the nurse case manager, the patient again logs on to the virtual office site with the patient browser 14 and retrieves the posted message at block 200. At block 202, the patient determines if the physician has provided a sufficient clinical response or other information to conclude the virtual office visit. If no, the patient may return to block 174 and post another message and the process repeats.

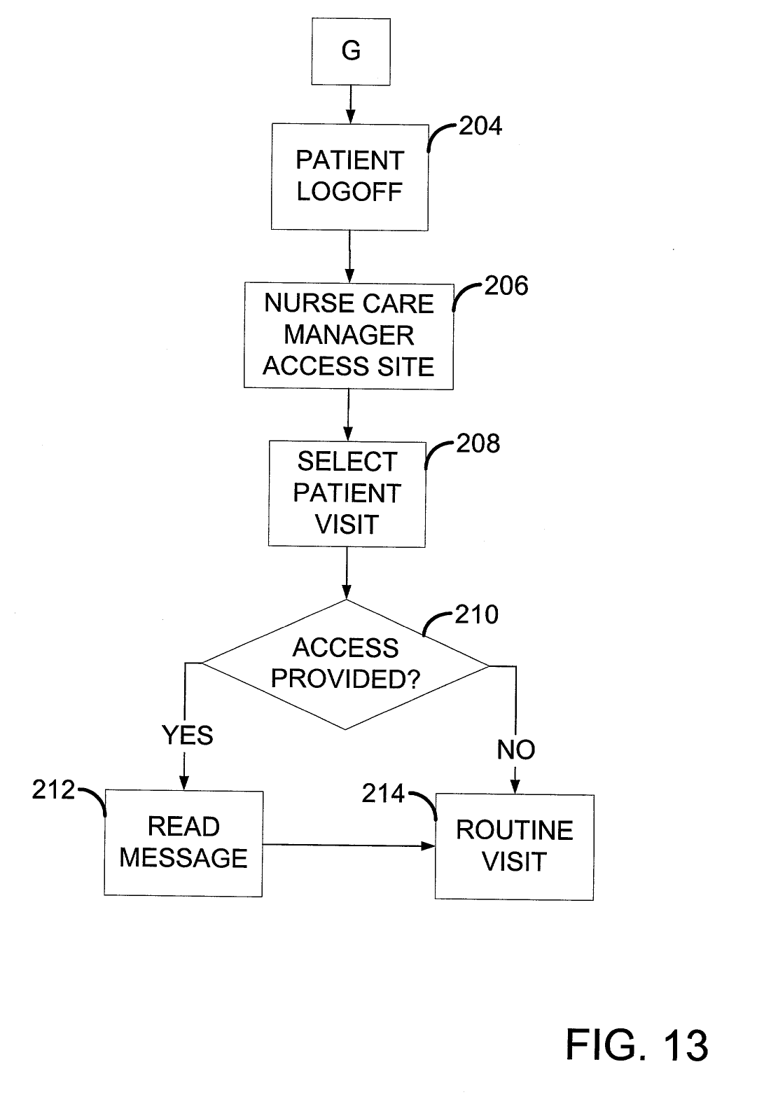

Referring now to FIG. 13, if the patient is satisfied with the physician's response and deems the virtual office visit concluded, at block 190 the patient logs off the virtual office site at block 204. At block 206, the nurse case manager logs on to the virtual office site with the nurse case manager browser 18. The nurse case manager accesses the patient information from the database 24 at block 206. At block 208, the nurse case manager selects the virtual office visit initiated by the patient and views the message headers. Determination of whether the nurse case manager has access to the contents of the messages is determined at block 210. If the nurse case manager has access, at block 212, the nurse case manager selects and reads the content of the messages to understand the current status of the patient's disease. At block 214, the nurse case manager performs a routine visit with the patient to assist in management of the patient's disease. The routine visit includes discussion of the contents of the messages. If the nurse case manager was not provided access to the content of the messages, the nurse case manager performs a routine visit with the patient to assist in managing the patient's disease at block 214.

The previously described embodiments of the outpatient treatment system 10 provide an efficient and effective mechanism for communication between a patient and the patient's physician. Those patients and physicians with established relationships are provided a secure, convenient and effective system for expanded maintenance of the patient's chronic health condition. The significantly greater flexibility in communications between the physician and the patient, along with the managerial oversight of the patient by the nurse case manager may provide the patient with better mastery and compliance with the care plan prescribed by the patient's physician. In addition, electronic payment request submission by the physicians, and access by physicians, patients and nurse case managers to current as well as previous communications, provides a convenient and efficient way to document the ongoing patient/physician relationship.

While the invention has been described above by reference to various embodiments, it will be understood that many changes and modifications can be made without departing from the scope of the invention. It is therefore intended that the foregoing detailed description be understood as an illustration of the presently preferred embodiments of the invention, and not as a definition of the invention. It is only the following claims, including all equivalents, that are intended to define the scope of this invention.

1. A method of providing outpatient treatment of a patient diagnosed with a chronic health related condition, the method comprising:
linking in a database a patient and a physician that are involved in a pre-existing patient/physician relationship, and that have been pre-approved as eligible to participate in a virtual office visit, the patient and the physician linked to define between whom communication will occur, the database being accessible by a server;
the server providing secure communication over a network between the patient and the physician treating the patient;
in the database -associating with the patient a diagnosis template specifically designed for a chronic health related condition suffered by the patient, the chronic health related condition previously diagnosed in accordance with the pre-existing patient/physician relationship:
in the database assigning a nurse case manager to the pre-existing patient/physician relationship to assist the patient in management of the predetermined chronic health related condition;
the server posting information entered by the patient with the diagnosis template to initiate a virtual office visit with the physician, wherein the diagnosis template is specific to the chronic health related condition of the patient and is displayable in response to the patient being granted secure access;
the server posting a response from the physician as part of the virtual office visit;
the patient and the physician continuing a dialog via posted messages until the virtual office visit is concluded;
the server granting access by the nurse case manager to the posted messages of the patient and the physician so that the nurse case manager can monitor the dialog:
the server receiving from the patient a selection of a level of access by the nurse case manager to view the posted messages of the patient and the physician the selection stored in the database: and the server submitting directly to a benefits provider of the patient over the network an electronic payment request for payment of the physician for the virtual office visit.

2.-3. (canceled)

4. The method of claim 1, wherein posting information entered by the patient with the diagnosis template comprises the patient selecting interactively from the diagnosis template symptoms being experienced, and the diagnosis template automatically updating to request additional information in response to patient selection of particular symptoms.

5. The method of claim 1, further comprising posting information from the patient and the physician as part of the virtual office visit only until the virtual office visit is concluded as indicated by the physician.

6. The method of claim 1, wherein submitting directly to a benefits provider of the patient over the network an electronic payment request comprises;
confirming a request to generate the electronic payment request in response to selection by the physician of a submit electronic payment request selector; and
producing a request confirmation comprising identification of the patient, the party to whom the request is directed to, and other information pertinent to the transaction.

7. The method of claim 1, wherein submitting an electronic payment request comprises directing the electronic payment request to a health benefits administrator by selection of a submit electronic payment request selector.

8. The method of claim 1, farther comprising maintaining a history in the database of user access of posted information.

9. A method of providing outpatient treatment to a patient diagnosed with a chronic health related condition, the method comprising:
in a database identifying a link between a patient with a chronic condition and a physician treating the patient, both pre-screened to be compatible with outpatient treatment over a network and involved in a pre-existing patient/physician relationship;
granting the patient secure access to a server located on the network that is in communication with the database and is responsive to authentication of the patient;
storing in the database an association of a diagnosis template with the patient, the diagnosis template specifically designed for the chronic health related condition of the patient, the diagnosis template generated with the server in response to the patient being authenticated to have secure access to the server, the diagnosis template accessible via the server by the patient as a displayable interactive display screen to initiate a dialog with the physician regarding a change in, or a current concern with, the chronic health related condition of the patient;
the server posting data entered by the patient with the diagnosis template;
during data entry, the server interactively configuring the content of the displayable interactive display screen of the diagnosis template being viewed by the patient in response to data entered by the patient;
providing the physician secure access to the server over the network with ability to view the data from the patient and participate in a dialog with the patient;
the server posting a clinical response from the physician, the clinical response responsive to the change or the concern raised by the patient, and accessible by the patient with the server; and
the server granting access by a nurse case manager to the dialog between the patient and the physician to assist the patient in managing the chronic health related condition,
the nurse case manager assigned to the patient as a precondition of the patient being provided secure access to the server located on the network, the nurse case manager being granted access after being authenticated as authorized by the server to view the dialog between the patient and the physician.

10. The method of claim 9, further comprising storing the data and the clinical response in the database on the server.

11. (canceled)

12. The method of claim 9, wherein the diagnosis template comprises at least one of a diabetes template, an asthma template and a congestive heart disease template.

13. The method of claim 9, wherein storing in the database an association of a diagnosis template, farther comprises storing in the database association of a plurality of diagnosis templates with the patient diagnosed with a plurality of chronic conditions, the associated diagnosis templates selectable by the patient to enter data as a function of the chronic conditions.

14. The method of claim 9, further comprising creating a virtual office visit within the server as a function of data within the diagnosis template.

15. The method of claim 14, farther comprising associating the clinical response with the virtual office visit.

16. The method of claim 14, further comprising:
posting a message on the server from the patient responsive to the clinical response; and
associating the posted message with the virtual office visit.

17. A method of providing outpatient treatment of a patient diagnosed with a chronic health related condition, the method comprising:
granting access by the patient and a physician treating the patient to a virtual office site over a network:;
eliciting communication between the patient and the physician over the network as a function of a virtual office visit initiated by the patient with the virtual office site;
posting a message with the virtual office site from the patient to the physician pertaining to the virtual office visit in response to receipt of data entry from the patient;
posting a clinical response from the physician responsive to the message from the patient with the virtual office site in response to receipt of data entry from the physician;
selectively granting access by a nurse case manager to review communications between the patient and the physician that involve the chronic health related condition of the patient in order for the nurse case manager to assist the patient with management of the chronic health related condition;
receiving from the nurse case manager selections from a list of predetermined intervention guidelines and selections of associated specific variables related to the chronic health related condition of the patient and the virtual office visit;
during the virtual office visit, the virtual office site monitoring activities of the patient and the physician within the virtual office site in accordance with the selected predetermined intervention guidelines to determine if the associated specific variables have changed;
the virtual office site alerting the nurse case manager with an alert message when predetermined thresholds are reached for any of the associated specific variables; and
transmitting over the network an electronic payment request to a health benefits administrator with the virtual office site as a function of the virtual office visit, in response to receipt from the physician that the virtual office visit is complete, and receipt of a selection by the physician of a submit electronic payment request selector.

18. (canceled)

19. The method of claim 17, further comprising granting the nurse case manager a level of access to communications between the patient and the physician, the level of access selected by the patient with the virtual office site.

20. The method of claim 17,
wherein allowing access comprises associating with the patient a diagnosis template specific to the chronic health related condition of the patient with the virtual office site; and
wherein eliciting communication comprises initiating the virtual office visit as a function of information entered by the patient in the diagnosis template.

21. The method of claim 17, wherein alerting the nurse case manager comprises generating notification to the nurse case manager with the virtual office site when a predetermined number of virtual office visits are initiated within a predetermined period of time.

22. The method of claim 17, wherein posting a message with the virtual office site from the patient comprises generating notification with the virtual office site directed to the physician.

23. The method of claim 22, wherein generating notification with the virtual office site directed to the physician comprises sending an e-mail notification to the physician.

24. The method of claim 17, wherein posting a clinical response from the physician with the virtual office site comprises generating notification with the virtual office site directed to the patient.

25. A method of providing outpatient treatment of a patient diagnosed with a chronic health related condition, the method comprising:
performing an approval process of a patient and a patients physician to ensure compatibility with outpatient treatment over a network;
when approved, linking the patient and the patient's physician in a database to establish secure communications on the network, to identify the previously established relationship of the patient and the patient's physician, and to enable private communication and dialog between the patient and the patient's physician;
in the database associating the patient with a diagnosis template designed for the chronic health related condition suffered by the patient, the diagnosis template generated for display to the patient when the patient is authenticated;
posting information entered by the patient into the diagnosis template to initiate a virtual office visit with the physician, the information entered into the diagnostic template by the patient and received over the network;
posting a response entered by the physician over the network as part of the virtual office visit, the response responsive to the information from the patient;
directing an electronic payment request to a health benefits administrator as a function of the virtual office visit;
granting selective access to the private communication between the physician and the patient that occurs during the virtual office visit, the selective access granted to a nurse case manager assigned to the patient during the approval process to assist the patient in management of the chronic health related condition and communication with the physician during the virtual office visit;
monitoring the private communication during the virtual office visit in accordance an intervention guideline, wherein the intervention guideline is configured to be indicative of a predetermined threshold being reached for pre-specified variables pertaining to the chronic health related condition of the patient and the virtual office visit, the variables pre-specified by the nurse case manager to be indicative of a potential issue related to the virtual office visit; and
transmitting an alert to the nurse case manager when the predetermined threshold is reached.

26. (canceled)

27. The method of claim 25, further comprising expanding the virtual office visit to display a visit header that includes the name of the patient, the name of the physician, the date of the initiation of the virtual office visit, the date of the last message posted pertaining to the virtual office visit, and the subject of the virtual office visit.

28. The method of claim 27, further comprising expanding the visit header to display a message header that includes contrasting visual notation indicative of the information posted by the patient and the response posted by the physician.

29. The method of claim 27, wherein providing selective access to the virtual office visit comprises preventing the nurse case manager access to the contents of the information and the response.

30. The method of claim 27, wherein the patient is diagnosed with a plurality of chronic conditions and linking the patient and the physician comprises linking the patient with a physician in the database for each of the chronic conditions.

31. An outpatient treatment system for treating a patient diagnosed with a chronic health related condition using a network, the outpatient treatment system comprising:
a server computer coupled to the network;
a database in communication with the server computer, the database configured to store a link between a patient and a physician that are involved in a pre-existing patient/physician relationship, and that have been pre-approved as eligible to participate in a virtual office visit, the patient and the physician linked to define between whom communication will occur;
a first browser coupled with the network, the first browser operable by the patient to communicate with the server computer;
a second browser coupled with the network, the second browser operable by the physician treating the patient to communicate with the server computer, the server computer responsive to communication from the first and second browsers to post messages from the patient and the physician that enable a dialog between the patient and the physician regarding a change in, or a concern with, the chronic health related condition of the patient; and
a third browser coupled with the network, the third browser operable by a nurse case manager, the server responsive to the third browser to allow selective access to posted messages of the patient and the physician by the nurse case manager to assist in management of the patient's health care, to act as an advocate for the patient, and to assist the patient in properly managing the chronic health related condition;
the server computer is operable to maintain the posted messages and related activity as a secure, separate, and distinct office visit conductible by the physician and representative of an exchange of messages between the patient and the physician based on a pre-existing relationship of the patient and the physician;
the server computer is further operable to monitor the exchange of messages in accordance with an intervention guideline, wherein the intervention guideline is configured to be indicative of a predetermined threshold being reached for pre-specified variables related to the exchange of messages between the patient and the physician, the intervention guidelines being configurable by the nurse case manager to be applied to the exchange of messages between the patient and the physician; and
the server computer is further operable to submit over the network an electronic payment request to a health benefits administrator of the patient in response to selection of a submit electronic payment request selector by the physician.

32. The outpatient treatment system of claim 31, wherein the server computer is operable to form a virtual office site, the virtual office site providing a secure site for the virtual office visit between the patient and the physician.

33. The outpatient treatment system of claim 32, wherein the server computer is operable to store posted messages of the virtual office visit in the database.

34. (canceled)

35. The outpatient treatment system of claim 31, wherein the server computer is operable to restrict access by the third browser to posted messages as a function of selection of an access level by the patient.

36. The outpatient treatment system of claim 31, wherein the server computer is operable to provide a diagnosis template to the first browser, the diagnosis template associated with the patient in the database based on the chronic health related condition of the patient.

37. The outpatient treatment system of claim 36, wherein the diagnosis template comprises at least one of a diabetes diagnosis template, an asthma diagnosis template and a congestive heart disease diagnosis template.

38. The outpatient treatment system of claim 31, wherein the server computer is operable to generate notification of posted messages.

39. (canceled)

40. The method of claim 25, wherein one of the pre-specified variables is a determined period of time in which posted information is to be read by the physician.

41. The method of claim 25, wherein one of the pre-specified variables is a threshold number of virtual office visits initiated by the patient within a determined period of time.
